(19) United States
(12) Patent Application Publication
(10) Pub. No.: US 2021/0059967 A1
(43) Pub. Date: Mar. 4, 2021

(54) LIQUID PARENTERAL COMPOSITIONS OF LEVOTHYROXINE

(71) Applicant: FTF PHARMA PRIVATE LIMITED, Sanand (IN)

(72) Inventors: Kinjal PARIKH, Anand (IN); Malay PATEL, Thaltej (IN); Jayanta Kumar MANDAL, Ahmedabad (IN)

(73) Assignee: FTF PHARMA PRIVATE LIMITED, Sanand (IN)

(21) Appl. No.: 16/550,677

(22) Filed: Aug. 26, 2019

(51) Int. Cl. A61K 31/198; A61K 47/18; A61K 47/12; A61K 9/00; A61K 47/02; A61K 47/10

(52) U.S. Cl. CPC A61K 31/198; A61K 47/183; A61K 47/10; A61K 9/0029; A61K 47/02; A61K 47/12

(57) ABSTRACT

Levothyroxine sodium for injection is particularly useful when thyroid replacement is needed on an urgent basis, for short term thyroid replacement, and/or when oral administration is not possible, such as for a patient in a state of myxedema coma. The present invention is therefore related to the liquid parenteral compositions comprising Levothyroxine or its pharmaceutically acceptable salt. In particular, the present invention describes ready to dilute liquid compositions which may be diluted using suitable diluent before administering to the patient in need thereof.

### FIELD OF INVENTION

The present invention relates, in general to the pharmaceutical field, and more precisely it relates to the pharmaceutical composition comprising Levothyroxine or its pharmaceutically acceptable salt. In particular, the present invention relates to the ready to dilute liquid compositions suitable for parenteral administration comprising Levothyroxine or its pharmaceutically acceptable salt.

### BACKGROUND OF THE INVENTION

A healthy thyroid produces hormones that regulate multiple metabolic processes and that play important roles in growth and development, in maturation of the central nervous system and bone including augmentation of cellular respiration and thermogenesis, and in metabolism of proteins, carbohydrates and lipids. The thyroid accomplishes its regulation functions by producing the hormones L-triiodothyronine (liothyronine; T3) and L-thyroxine (levothyroxine; T4).

Thyroxine active drugs are known for both therapeutic and prophylactic treatment of thyroid disorders. The thyroid accomplishes its regulation functions by producing the hormones L-triiodothyronine (liothyronine; T3) and L-thyroxine (levothyroxine; T4). The physiological actions of thyroid hormones are produced predominantly by T3, the majority of which (approximately 80%) is derived from T4 by de-iodination in peripheral tissues.

Thyroid hormones are believed to exert their physiologic actions through control of DNA transcription and protein synthesis. It is presently believed that the T3 and T4 hormones diffuse into the cell nucleus and bind to thyroid receptor proteins attached to DNA. This hormone nuclear receptor complex then activates gene transcription and synthesis of messenger RNA and cytoplasmic proteins. The physiological actions of thyroid hormones are believed to be produced predominantly by T3, approximately 80% of which is derived from T4 by de-iodination in peripheral tissues.

Both T3 and T4 are stored in the thyroid as thyroglobulin adducts with serum proteins. Once secreted by the thyroid, T3 and T4 primarily exist in the circulatory system as their thyroglobulin adducts, and are in equilibrium with small amounts (<1%) of the unbound hormones, which are the metabolically active species. T4 has higher serum levels, slower metabolic clearance, and a longer half-life than T3, which may be due to the higher affinity of serum proteins for T4 compared to T3.

Administration of levothyroxine sodium provides T4 to a patient. Once absorbed, the administered T4 behaves identically to T4 that otherwise would be secreted by the thyroid gland of the patient, and binds to the same serum proteins, providing a supply of circulating T4-thyroglobulin in the patient. The administered T4 may be de-iodinated in vivo to T3. As a result, a patient receiving appropriate doses of levothyroxine sodium will exhibit normal blood levels of T3, even when the patient's thyroid gland has been removed or is not functioning.

A patient whose thyroid gland has been removed, or whose thyroid gland functions at an undesirably low level (hypothyroidism), may be treated by administration of a daily maintenance dose of 50-100 micrograms (μg) of levothyroxine sodium. A patient in need of additional intervention may be treated by administration of an initial dose of 200-500 μg or 300-500 μg of levothyroxine sodium and/or with a 2^{nd} day dose of 100-300 μg of levothyroxine sodium. Formal names for levothyroxine sodium include 4-(4-hydroxy-3,5-diiodophenoxy)-3,5-diiodo-L-phenylalanine sodium, and L-tyrosine-O-(4-hydroxy-3,5-diiodophenyl)-3,5-diiodo-monosodium salt. The structural formula is:

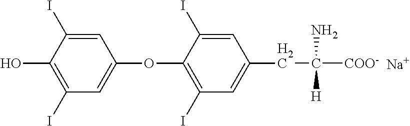

Available oral dosage forms of Levothyroxine include capsules, tablets, and oral solutions. Oral solutions are preferred over other solid oral dosage forms because of their use in pediatric and geriatric patients who may have difficulty to swallow tablets or capsules. Unfortunately, solutions of Levothyroxine are less stable compared to tablets during storage. Also, Levothyroxine solutions may comprise relatively high amounts of liothyronine, which is believed to be the source of side-effects in certain patients. Aqueous Levothyroxine solutions are prone to decomposition compared to the solid forms. Therefore our research group as a part of their earlier research developed a process for preparing oral solutions of Levothyroxine possessing enhanced stability. Such an invention has been disclosed and claimed in our earlier patent application published internationally WO 2018069805.

Levothyroxine sodium for injection is particularly useful when thyroid replacement is needed on an urgent basis, for short term thyroid replacement, and/or when oral administration is not possible, such as for a patient in a state of myxedema coma. Levothyroxine sodium for injection is available as sterile lyophilized product for parenteral administration containing 100 meg/vial, 200 mcg/vial and 500 meg/vial. Conventional formulations of levothyroxine sodium for injection are preservative-free lyophilized powders containing synthetic crystalline levothyroxine sodium, mannitol, tribasic sodium phosphate, and sodium hydroxide. These conventional formulations typically contain 10 mg mannitol, 700 meg of tribasic sodium phosphate and 100 meg or 200 meg or 500 meg of Levothyroxine sodium. Administration of the conventional formulation involves reconstitution of the lyophilized powder in 5 mL of 0.9% sodium chloride injection, to provide injectable solutions having levothyroxine sodium concentrations of 20 mcg/mL, 40 mcg/mL or 100 mcg/mL.

WO 2017013591 (filed by Leiutis Pharmaceuticals) incorporated herein by reference in its entirety, discloses liquid parenteral formulations of Levothyroxine comprising buffering agents, one or more solvents, with or without stabilizing agents and/or solubilizing agents and optionally one or more pharmaceutically acceptable excipients selected from pH adjusting agents and anti-oxidants. The stabilizing agents used in the formulations of WO 2017013591 publication include sodium iodide, potassium iodide and the like.

The solubilizing agents used in the formulations of WO 2017013591 publication include cyclodextrins such as α, β and γ-cyclodextrin and cyclodextrins modified with alkyl-, hydroxyl-alkyl-, dialkyl-, and sulfoalkyl-ether modified cyclodextrins such as methyl or hydroxypropyl β-cyclodexrins (HPβCD), methyl-and-ethyl-β-cyclodextrin, sulfoalkylether-substituted beta-cyclodextrin, sulfobutylether-β-cyclodextrin (SBECD) and the like.

The buffering agents suitable for use in the formulations of WO2017013591 publication are amino acids such as arginine, alanine, histidine, glycine and lysine; citrate, glutamate, bicarbonate, tartrate, benzoate, lactate, gluconate, TRIS, acetate, meglumine, borate and phosphate buffer.

The solvents suitable for use in the formulations of WO 2017013591 publication are dimethylacetamide, dimethyl sulfoxide, N-methylpyrrolidone, dimethylisosorbide, ethanol, propylene glycol, polyethylene alcohol, propylene glycol esters, polyethylene glycols, glycerin, water and the like. According to the WO 2017013591 publication preferred solvents are water and propylene glycol.

The pH adjusting agents suitable for use in the formulations of WO 2017013591 publication are sodium hydroxide, sodium carbonate, sodium bicarbonate, potassium hydroxide, ammonium carbonate, hydrochloric acid, citric acid, lactic acid, phosphoric acid, sodium phosphate, sulfuric acid and the like.

WO 2017013591 publication also teaches that the formulations disclosed therein may also contain one or more anti-oxidants such as sodium sulfite, sodium bisulfite, sodium metabisulfite, sodium thiosulfate, sodium formaldehyde sulfoxylate, citric acid, tocopherol, butylated hydroxyl anisole, butylated hydroxyl toluene, monothioglycerol, ascorbic acid, sodium ascorbate and propyl gallate.

Further, WO 2017013591 publication teaches preferred formulation of Levothyroxine comprising:
i. Levothyroxine sodium;
ii. Stabilizing agents and/or solubilizing agents selected from sodium iodide, potassium iodide and cyclodextrins;
iii. Buffering agent(s) selected from aminoacids such as arginine, alanine, histidine, glycine and lysine; citrate, glutamate, bicarbonate, tartrate, benzoate, lactate, gluconate, TRIS, acetate, borate and phosphate buffer;
iv. One or more solvents selected from the group comprising water, polyethylene glycol, ethanol, propylene glycol and glycerin;
v. Optionally one or more pharmaceutically acceptable excipients selected from pH adjusting agents and anti-oxidants.

However, upon reading specification of WO 2017013591 it seems that a solubilizing agent especially sulfobutylether-beta-cyclodextrin (SBECD) and the like is an essential component of the compositions disclosed in WO 2017013591.

U.S. Pat. Nos. 9,006,289, 9,168,238 and 9,168,239 (assigned to Fresenius Kabi USA, LLC), incorporated herein by reference in its entirety, discloses lyophilized solid compositions comprising Levothyroxine sodium, a buffer and mannitol.

U.S. Pat. No. 9,782,376 (assigned to Fresenius Kabi USA, LLC), incorporated herein by reference in its entirety, discloses ready to use injectable formulation of Levothyroxine sodium comprising tromethamine, sodium iodide, sodium chloride and water. Upon reading complete specification of U.S. Pat. No. 9,782,376 it seems that tromethamine, i.e. base is an essential component of the formulations disclosed in U.S. Pat. No. 9,782,376.

US 20180153838 (filed by Fresenius Kabi USA. LLC), incorporated herein by reference in its entirety, discloses a ready to use liquid formulation comprising levothyroxine or a pharmaceutically acceptable salt thereof, a stabilizing agent comprising tromethamine, not more than 2% liothyronine (T3) and water. The said formulation is stable for at least 12 months at 25±2° C. The stabilizing agents taught in US 20180153838 further include bis(2-hydroxyethyl)-imino-tris(hydroxymethyl)methane, monoethanolamine, diethanolamine, triethanolamine, 2-amino-2-methyl-1,3-propanediol, 2-dimethylamino-2-methyl-1-propanediol, 2-amino-2-ethylpropanol, 2-amino-1-butanol, and 2-amino-2-methyl-1-propanol or salt of iodide such as sodium iodide or potassium iodide. Upon reading complete specification of US 20180153838 it seems that tromethamine, i.e. base is an essential component of the formulations disclosed in US 20180153838.

WO 2019023791 (filed by Apollo Pharmaceuticals, Inc.), incorporated herein by reference in its entirety, discloses an aqueous parenteral formulation comprising levothyroxine sodium, one or more antioxidants, one or more chelating agents, one or more buffering agents, one or more pH adjusting agents and one or more solvents.

Levothyroxine has extremely short stability, worsened under conditions of high humidity and temperature. Due to this instability, Levothyroxine injectable formulations a used in the form of lyophilized formulations that are dissolved in 0.9% sodium chloride injection immediately before injection. Further, due to this instability, other liquid parenteral formulations of Levothyroxine known in the art contains a stabilizing agent, a solubilizing agent, a chelating agent or a combination thereof which provides the formulation desired stability.

Levothyroxine sodium is quite unstable, hygroscopic and degrades rapidly when subjected to high humidity, light or high temperature. Degradation is further enhanced by the presence of water. Hence, attempts to develop an intravenous preparation of Levothyroxine were limited. Therefore looking at the existing need in the art, it is desirable to have liquid parenteral compositions of Levothyroxine or its pharmaceutically acceptable salt having prolonged stability profile.

The inventors of the present invention, as apart of their continuous research, have now developed parenteral compositions of Levothyroxine which are ready to dilute, concentrate liquids comprising one or more solvents/co-solvents and optionally one or more pharmaceutically acceptable excipients selected from the group comprising of antioxidants, pH adjusting/modifying agents, preservatives and the like or any combinations thereof. The liquid compositions of the invention may also comprise water (e.g. water for injection) as a vehicle or carrier. The liquid compositions of the invention may further comprise one or more pharmaceutically acceptable excipients selected from stabilizing agents, chelating agents and combinations thereof. Surprisingly, the liquid compositions of the present invention show desired stability profile even in the absence of one or more pharmaceutically acceptable excipients described in the prior art documents.

### SUMMARY OF THE INVENTION

Parenteral compositions of Levothyroxine are useful when the patients cannot tolerate oral therapy or when oral administration is not possible. Daily administration of Levothyroxine Sodium for Injection may be maintained until the patient is capable of tolerating an oral dose and is clinically stable. It one embodiment, the present invention provides parenteral compositions of Levothyroxine or its pharmaceutically acceptable salt.

Liquid concentrate compositions are useful because of small quantities of liquid solubilizers/solvents/co-solvents, vehicles or carriers. Such concentrate compositions are contained in small vials which are easy to handle and transport. Therefore, in another embodiment, the present invention provides liquid concentrate compositions which are ready to dilute using a suitable diluent before administering to the patient in need thereof.

Ready to use compositions are useful because they do not require dilution prior to administration to the patients. Ready to use compositions eliminate possibilities of dosage errors while diluting the concentrate liquids. Therefore, in another embodiment, the present invention provides ready to use liquid compositions which can be administered to the patient in need thereof without diluting further.

In another embodiment, the present invention provides liquid compositions comprising Levothyroxine or its pharmaceutically acceptable salt and one or more solvents/co-solvents. In another embodiment, the liquid compositions may further comprise one or more pharmaceutically acceptable excipients selected from the group comprising of antioxidants, pH adjusting/modifying agents, preservatives and the like or a combinations thereof. In another embodiment, the liquid compositions of the present invention optionally comprise a buffering agent. In another embodiment, the liquid compositions of the present invention may further comprise water (e.g. water for injection) as a vehicle or carrier. In another embodiment, the liquid compositions of the present invention may further comprise one or more pharmaceutically acceptable excipients selected from stabilizing agents, chelating agents and combinations thereof. The liquid compositions of the present invention are stable under storage conditions.

In another embodiment, the liquid compositions may further comprise one or more pharmaceutically acceptable excipients suitable for preparing parenteral or injectable compositions.

In another embodiment, the present invention provides a process for the preparation of the liquid compositions of Levothyroxine or its pharmaceutically acceptable salt.

In another embodiment, the present invention provides use the parenteral compositions of the present invention for thyroid hormone replacement therapy in cases of reduced or absent thyroid function e.g., ailments such as myxedema, cretinism and obesity. Parenteral compositions of the present invention are particularly useful when thyroid replacement is needed on an urgent basis, for short term thyroid replacement, and/or when oral administration is not possible.

### DETAILED DESCRIPTION OF THE INVENTION

Prior art documents disclose parenteral compositions of Levothyroxine which include solid lyophilized compositions of Levothyroxine as well as liquid parenteral formulations of Levothyroxine. As described in the foregoing paragraphs, liquid parenteral compositions are especially useful when patients are not capable of taking oral therapy or when oral administration is not possible such as for a patient in a state of myxedema coma. Therefore, in one of the principal embodiments of the present invention, parenteral compositions comprising Levothyroxine or its pharmaceutically acceptable salt are described.

According to one of the further embodiments of the present invention, the liquid compositions of the present invention comprise Levothyroxine or its pharmaceutically acceptable salt and one or more solvents/co-solvents. In one of the further embodiments of the present invention, the liquid compositions may further comprise one or more pharmaceutically acceptable excipients selected from the group comprising of antioxidants, buffering agents, pH adjusting/modifying agents, preservatives and the like or a combination thereof. In one of the further embodiments of the present invention, the liquid compositions may further comprise water (e.g. water for injection) as vehicle or carrier. In one of the further embodiments of the present invention, the liquid compositions may further comprise one or more pharmaceutically acceptable excipients selected from stabilizing agents, chelating agents and combinations thereof. In one of the further embodiments of the present invention, the liquid compositions optionally comprise one or more buffering agents. In one of the further embodiments of the present invention, the liquid compositions may further comprise one or more pharmaceutically acceptable excipients suitable for preparing parenteral or injectable compositions.

In one embodiment, the present invention provides liquid compositions of Levothyroxine comprising,
(i) levothyroxine or its pharmaceutically acceptable salt, and
(ii) one or more solvent(s)/co-solvent(s).

In another embodiment, the present invention provides liquid compositions of Levothyroxine comprising,
(i) levothyroxine or its pharmaceutically acceptable salt,
(ii) one or more solvent(s)/co-solvent(s), and
(iii) one or more buffering and/or pH adjusting/modifying agent(s).

In another embodiment, the present invention provides liquid compositions of Levothyroxine comprising,
(i) levothyroxine or its pharmaceutically acceptable salt,
(ii) one or more solvent(s)/co-solvent(s),
(iii) one or more buffering and/or pH adjusting/modifying agent(s), and (iv) one or more vehicle(s)/carrier(s).

In another embodiment, the present invention provides liquid compositions of Levothyroxine comprising,
(i) levothyroxine or its pharmaceutically acceptable salt,
(ii) one or more solvent(s)/co-solvent(s),
(iii) one or more buffering and/or pH adjusting/modifying agent(s),
(iv) one or more preservative(s), and
(v) one or more vehicle(s)/carrier(s).

In another embodiment, the present invention provides liquid compositions of Levothyroxine comprising,
(i) levothyroxine or its pharmaceutically acceptable salt,
(ii) one or more solvent(s)/co-solvent(s),
(iii) one or more antioxidant(s),
(iv) one or more buffering and/or pH adjusting/modifying agent(s), and
(v) one or more vehicle(s)/carrier(s).

In another embodiment, the present invention provides liquid compositions of Levothyroxine comprising,
(i) levothyroxine or its pharmaceutically acceptable salt,
(ii) one or more solvent(s)/co-solvent(s),
(iii) one or more antioxidant(s),
(iv) one or more pH adjusting/modifying agent(s), and
(v) one or more vehicle(s)/carrier(s).

In another embodiment, the present invention provides liquid compositions of Levothyroxine comprising,
(i) levothyroxine or its pharmaceutically acceptable salt,
(ii) one or more solvent(s)/co-solvent(s),
(iii) one or more antioxidant(s),
(iv) one or more buffering and/or pH adjusting/modifying agent(s),
(v) one or more preservative(s), and
(vi) one or more vehicle(s)/carrier(s).

In another embodiment, the present invention provides liquid compositions of Levothyroxine comprising,
(i) levothyroxine or its pharmaceutically acceptable salt,
(ii) a solvent/co-solvent selected from glycerin,
(iii) an antioxidant selected from citric acid,
(iv) a pH adjusting agent selected from sodium hydroxide, and
(v) a vehicle/carrier selected from water for injection.

In another embodiment, the present invention provides liquid compositions of Levothyroxine comprising,
(i) levothyroxine or its pharmaceutically acceptable salt,
(ii) one or more solvent(s)/co-solvent(s),
(iii) one or more antioxidant(s),
(iv) one or more stabilizing agent(s),
(v) one or more chelating agent(s).
(vi) one or more pH adjusting agent(s), and
(vii) one or more vehicle(s)/carrier(s).

In another embodiment, the present invention provides liquid compositions of Levothyroxine comprising,
(i) levothyroxine or its pharmaceutically acceptable salt,
(ii) one or more solvent(s)/co-solvent(s),
(ii) one or more antioxidant(s),
(iv) one or more stabilizing agent(s),
(v) one or more pH adjusting agent(s), and
(vi) one or more vehicle(s)/carrier(s).

In another embodiment, the present invention provides liquid compositions of Levothyroxine comprising,
(i) levothyroxine or its pharmaceutically acceptable salt,
(ii) one or more solvent(s)/co-solvent(s),
(iii) one or more antioxidant(s),
(iv) one or more chelating agent(s),
(v) one or more pH adjusting agent(s), and
(vi) one or more vehicle(s)/carrier(s).

In another embodiment, the present invention provides liquid compositions of Levothyroxine comprising,
(i) levothyroxine or its pharmaceutically acceptable salt,
(ii) a solvent/co-solvent selected from glycerin,
(iii) an antioxidant selected from citric acid,
(iv) a stabilizing agent selected from sodium iodide,
(v) a chelating agent selected from disodium EDTA,
(vi) a pH adjusting agent selected from sodium hydroxide, and
(vii) a vehicle/carrier selected from water for injection.

In another embodiment, the present invention provides liquid compositions of Levothyroxine comprising,
(i) levothyroxine or its pharmaceutically acceptable salt,
(ii) a solvent/co-solvent selected from glycerin,
(iii) an antioxidant selected from citric acid,
(iv) a stabilizing agent selected from sodium iodide,
(v) a pH adjusting agent selected from sodium hydroxide, and
(vi) a vehicle/carrier selected from water for injection.

In another embodiment, the present invention provides liquid compositions of Levothyroxine comprising,
(i) levothyroxine or its pharmaceutically acceptable salt,
(ii) a solvent/co-solvent selected from glycerin,
(iii) an antioxidant selected from citric acid,
(iv) a chelating agent selected from disodium EDTA,
(v) a pH adjusting agent selected from sodium hydroxide, and
(vi) a vehicle/carrier selected from water for injection.

In another embodiment, the present invention provides liquid compositions of Levothyroxine comprising,
(i) levothyroxine or its pharmaceutically acceptable salt;
(ii) a solvent/co-solvent selected from glycerine;
(iii) a buffer selected from glycine;
(iv) a pH adjusting agent selected from sodium hydroxide; and
(v) a vehicle/carrier selected from water for injection.

In some embodiments, the liquid compositions of the present invention are ready to dilute liquid concentrate compositions. In some embodiments, the liquid compositions of the present invention are ready to use compositions.

In some of the embodiments, liquid Levothyroxine compositions as described above may further comprise other pharmaceutically acceptable excipients.

In some of the embodiments, the ready to dilute, liquid concentrate Levothyroxine compositions may not include a stabilizing agent. In some of the alternative embodiments, the ready to dilute, liquid concentrate Levothyroxine compositions may not include a stabilizing agent and a solubilizing agent. In some of the embodiments, the ready to dilute, liquid concentrate Levothyroxine compositions may not include a stabilizing agent, a solubilizing agent, a chelating agent and a combination thereof. The stabilizing agents, solubilizing agents and chelating agents excluded from the liquid compositions of the present invention are, for example, those described in the prior art documents mentioned in the foregoing paragraphs.

According to one of the further embodiments, the pH of the compositions of the present invention ranges between about 4.0 to about 12.0. According to one of the further embodiments, the pH of the compositions of the present invention ranges between about 5.5 and about 11.0. According to one of the further embodiments, the pH of the compositions of the present invention ranges between about 7.0 and about 11.0. According to one of the further embodiments, the pH of the compositions of the present invention ranges between about 8.0 and about 12.0. According to one of the further embodiments, the pH of the compositions of the present invention ranges between about 9.0 and about 12.0. According to one of the further embodiments, the pH of the compositions of the present invention ranges between about 10.0 and about 12.0.

In one of the further embodiments, the present invention provides process for the preparation of liquid compositions of Levothyroxine. The process comprises following steps:
(i) Take required quantity of one or more solvent(s)/co-solvent(s) in suitable vessel;
(ii) Optionally add required quantity of one or more buffering agent(s) and/or one or more pH adjusting/modifying agent(s) to adjust the pH between about 3.0 and about 5.0;
(iii) Add required quantity of Levothyroxine or its pharmaceutically acceptable salt;
(iv) Adjust the pH between about 8.0 and 11.0; and
(v) Adjust the volume using one or more solvent(s)/co-solvent(s) to the required batch size.

In one of the further embodiments, the present invention provides process for the preparation of liquid compositions of Levothyroxine. The process comprises following steps:
(i) Take required quantity of one or more solvent(s)/co-solvent(s) in suitable vessel;
(ii) Add required quantity of one or more buffering agent(s) to adjust the pH between about 3.0 and about 5.0;
(ii) Add required quantity of one or more pH adjusting/modifying agent(s) to adjust the pH between about 8.0 and about 11.0
(iv) Add required quantity of Levothyroxine or its pharmaceutically acceptable salt; and
(v) Adjust the volume using one or more solvent(s)/co-solvent(s) to the required batch size.

In one of the further embodiments, the present invention provides process for the preparation of liquid compositions of Levothyroxine. The process comprises following steps:
(i) Take required quantity of one or more solvent(s)/co-solvent(s) in suitable vessel;
(ii) Add required quantity of one or more vehicle(s)/carrier(s);
(iii) Add required quantity of one or more buffering agent(s) to adjust the pH between about 3.0 and about 5.0;
(iv) Add required quantity of one or more pH adjusting/modifying agent(s) to adjust the pH between about 8.0 and about 11.0;
(v) Add required quantity of Levothyroxine or its pharmaceutically acceptable salt; and
(vi) Adjust the volume using one or more vehicle(s)/carrier(s) to the required batch size.

In one of the further embodiments, the present invention provides process for the preparation of liquid compositions of Levothyroxine. The process comprises following steps:
(i) Take required quantity of one or more solvent(s)/co-solvent(s) in suitable vessel;
(ii) Add required quantity of one or more vehicle(s)/carrier(s);
(iii) Add required quantity of one or more preservative(s);
(iv) Add required quantity of one or more buffering agent(s) to adjust the pH between about 3.0 and about 5.0;
(v) Add required quantity of Levothyroxine or its pharmaceutically acceptable salt;
(vi) Add required quantity of one or more pH adjusting/modifying agent(s) to adjust the pH between about 8.0 and about 11.0; and
(vii) Adjust the volume using one or more vehicle(s)/carrier(s) to the required batch size.

In one of the further embodiments, the present invention provides process for the preparation of liquid compositions of Levothyroxine. The process comprises following steps:
(i) Mix required quantities of a solvent(s)/co-solvent(s) and a vehicle/carrier in a suitable vessel;
(ii) Add required quantity of an antioxidant in step (i);
(iii) Adjust the pH of step (ii) with a suitable pH adjusting agent between about 8.0 and about 12.0;
(iv) Add and dissolve required quantity of Levothyroxine or its pharmaceutically acceptable salt;
(v) Adjust the volume using a vehicle/carrier to the required batch size.

In one of the further embodiments, the present invention provides process for the preparation of liquid compositions of Levothyroxine. The process comprises following steps:
(i) Mix required quantities of a solvent(s)/co-solvent(s) and a vehicle/carrier in a suitable vessel;
(ii) Add required quantity of an antioxidant in step (i);
(iii) Adjust the pH of step (ii) with a suitable pH adjusting agent between about 8.0 and about 12.0;
(iv) Add and dissolve required quantity of a stabilizing agent in step (iii);
(v) Add and dissolve required quantity of Levothyroxine or its pharmaceutically acceptable salt; and
(vi) Adjust the volume using a vehicle/carrier to the required batch size.

In the above mentioned process, if a chelating agent is used in the levothyroxine composition, the same can be added in step (v) by preparing aqueous solution thereof.

In one of the further embodiments, the present invention provides process for the preparation of liquid compositions of Levothyroxine. The process comprises following steps:
(i) Mix required quantities of a solvent(s)/co-solvent(s) and a vehicle/carrier in a suitable vessel;
(ii) Add required quantity of an antioxidant in step (i);
(iii) Adjust the pH of step (ii) with a suitable pH adjusting agent between about 8.0 and about 12.0;
(iv) Add and dissolve required quantity of Levothyroxine or its pharmaceutically acceptable salt in step (iii);
(v) Add required quantity of separately prepared aqueous solution of a chelating agent in step (iv); and
(vi) Adjust the volume using a vehicle/carrier to the required batch size.

Those who are skilled in the art can understand that some variations in the above described processes can be adopted when one or more other pharmaceutically acceptable excipients are used. A skilled person can change and/or omit sequences of the steps of the above described processes for the purposes of suitability and convenience where one or more pharmaceutically acceptable excipients may or may not be used without affecting and diminishing the quality and characteristics of the resulting product. Such variations/changes/omissions/additions are well within the scope of the present invention.

The term “Levothyroxine” as used herein also includes, the pharmaceutically acceptable salts, solvates, hydrates and anhydrous form of Levothyroxine. In particular, the liquid compositions of the present invention comprise Levothyroxine or any pharmaceutically acceptable salt thereof. In one of the embodiments, the liquid compositions of the present invention comprise Levothyroxine sodium. In one of the preferred embodiments, the Levothyroxine sodium is levothyroxine sodium pentahydrate, which is the sodium salt of the levo-isomer of thyroxine, an active physiological substance found in the thyroid gland.

Vehicles may be used in the liquid compositions of the present invention. Vehicles are the liquid bases that carry drugs and other excipients in dissolved or dispersed state. Vehicles may be aqueous or non-aqueous or mixture thereof. Non-aqueous solvents may also be added in the liquid compositions of the present invention to increase the solubility of poorly soluble substances and enhance the chemical stability of a drug. Suitable solvents/co-solvents, that may be employed, in the liquid compositions of the invention include, but are not limited to dichloromethane, acetonitrile, ethyl acetate, acetone, propylene carbonate, water, glycerin, coconut fatty acid diethanolamide, medium and/or long chain fatty acids or glycerides, monoglycerides, diglycerides, triglycerides, structured triglycerides, soyabean oil, peanut oil, corn oil, corn oil mono glycerides, corn oil di glycerides, corn oil triglycerides, polyethylene glycol, caprylocaproymacroglycerides, caproyl 90, propylene glycol, propylene glycol esters, polyoxyethylenesorbitan fatty acid esters, polyoxyethylene castor oil derivatives, castor oil, cottonseed oil, olive oil, safflower oil, peppermint oil, coconut oil, palm seed oil, beeswax, oleic acid, methanol, ethanol, isopropyl alcohol, butanol, polyethylene alcohol, acetone, methyl isobutyl ketone, methyl ethyl ketone, N-methylpyrrolidone, dimethylacetamide, dimethylsulfoxide, dimethylisosorbide and the like or combinations thereof. In some of the embodiments of the present invention, the terms “solvents/co-solvents”, “solubilizers” and “vehicles” may be used interchangeably. In some of the preferred embodiments of the present invention, a solvent and a vehicle are two different ingredients.

In some of the preferred embodiments, a solvent/co-solvent used in the formulations of the present invention is glycerin and a vehicle or carrier used in the formulations of the present invention is water, suitably water for injection.

Buffering agents as used in the present invention without limitation include amino acids such as arginine, alanine, histidine, glycine and lysine; citrate, glutamate, bicarbonate, tartrate, benzoate, lactate, gluconate, triethanolamine, trolamine, acetate, meglumine, borate, phosphate, ammonium phosphate, diethanolamine, potassium acetate, potassium citrate, potassium metaphosphate, potassium phosphate, sodium acetate, sodium citrate, sodium glycolate, sodium lactate and sodium phosphate.

pH adjusting/modifying agents as used in the present invention without limitation include acetic acid, adipic acid, ammonium carbonate, ammonium hydroxide, boric acid, citric acid, fumaric acid, hydrochloric acid, malic acid, nitric acid, propionic acid, potassium bicarbonate, potassium chloride, sodium bicarbonate, sodium borate, sodium carbonate, sodium chloride, sodium hydroxide, sodium proprionate, succinic acid, sulfuric acid, tartaric acid, triethylamine. It may happen that an excipient which may be used as a buffering agent may also be used as a pH adjusting agent and vice versa.

A “Buffering agent” or a “pH adjusting agent” as used herein is a system which is used for the purposes and is capable of maintaining the desired/required pH of the formulations throughout desired/required time period, e.g. stability studies and/or shelf life of the drug product. The desired pH of the formulations according to the present invention is between about 8.0 and about 12.0.

In some of the embodiments of the invention, both buffering agent and pH adjusting agent are used. In some of the embodiments of the invention, only a buffering agent is used. In some of the embodiments of the invention, only a pH adjusting agent is used.

In some of the preferred embodiments, sodium hydroxide is used as a pH adjusting agent which adjusts and/or maintains the desired/required pH.

Microbiological contamination presents a significant health hazard in liquid formulations. Therefore, the use of preservatives become inevitable to prevent the growth of microorganisms during the product's manufacture and shelf life, although it may be most desirable to develop a “preservative-free” formulation to address the increasing concerns about the biological activity of these compounds. Most formulations require some kind of preservative to ensure no microbial growth. Non-limiting examples of preservatives are Alcohol, Ethanol, Chlorobutanol, Phenoxyethanol, Potassium benzoate, Benzyl alcohol, Benzoic acid, Potassium sorbate, Sorbic acid, Benzalkonium chloride, Benzethonium chloride. Cetrimonium bromide, Cetylpyridinium chloride, Bronopol, Chlorbutol, Chlorocresol, Cresol, Butylparaben, Methylparaben, Propylparaben, Ethylparaben, Phenol, Thymol, Phenylethanol, Sodium benzoate, Antimicrobial solvents like Propylene glycol, Glycerin, Chloroform and the like. In addition, some formulation ingredients like nonionic surfactants, quaternary ammonium compounds, gelatin, ferric salts, calcium salts and salts of heavy metals, including silver, lead, and mercury prevent microbial growth.

Antioxidants can be compounds that can reduce a drug that has been oxidized, or compounds that are more readily oxidized than the agents they are to protect (oxygen scavengers). Many of the lipid-soluble antioxidants act as scavengers. Antioxidants can also act as chain terminators, reacting with free radicals in solution to stop the free-radical propagation cycle. Non-limiting examples of anti-oxidants are α-Tocopherol acetate, Ascorbic acid, Citric acid, Erythorbic acid, Butylated hydroxytoluene (BHT), d-α-Tocopherol natural, Monothioglycerol, Thioglycolic acid, Sodium bisulfite, Sodium sulfite, Sodium metabisulfite, Potassium metabisulfite, Acetone sodium bisulfite, Ascorbyl palmitate, Cysteine, d-α-tocopherol synthetic, Nordihydroguaiaretic acid, Sodium formaldehyde sulfoxylate, Sodium thiosulfate. Acetylcysteine, Ascorbyl palmitate, Butylated hydroxyanisole (BHA), Cysteine hydrochloride, Dithiothreitol, Propyl gallate, Thiourea and the like. In some of the embodiments of the present invention, an antioxidant may be chosen in such a way so that it can also be used to adjust the desired pH of the formulations. Preferred antioxidant used in the formulations of the present invention is citric acid.

Surfactant is a general name for materials that possess surface activity; in solution they tend to orient at the surface of the liquid. There are several general classes of surfactants: anionic, cationic, amphoteric and non-ionic. Surfactants are amphiphilic molecules, i.e. part of the molecule is hydrophilic, and part is lipophilic. This combination of the two opposite affinities in the same molecule causes them to orient to the interface and thereby reduce the interfacial tension between the continuous and disperse phases, such as in emulsions and suspensions. Ionic surfactants work primarily through electrostatic forces, whereas non-ionic surfactants work primarily through steric forces. Non-limiting examples of surfactants are Sodium lauryl sulfate, Docusate sodium. Cocamidopropyl amino betaine, Polyoxyethylene sorbitan fatty acid esters (Polysorbate, Tween®), Polyoxyethylene 15 hydroxystearate (Macrogol 15 hydroxystearate, Solutol HS15®), Polyoxyethylene castor oil derivatives (Cremophor® EL, ELP, RH 40), Polyoxyethylene stearates (Myrj®), Sorbitan fatty acid esters (Span®), Polyoxyethylene alkyl ethers (Brij®), Polyoxyethylene nonylphenol ether (Nonoxynol®) and the like.

In some embodiments, the stabilizing agent is selected from sodium iodide, potassium iodide, povidone, povidone K12, povidone K17, crospovidone, sorbitol, and sorbitol solution. In some of the preferred embodiments, the stabilizing agent is sodium iodide.

In some embodiments, a chelating agent is selected from edetate disodium (disodium EDTA), edetate disodium (disodium EDTA) anhydrous, edetate sodium, edetate calcium disodium, edetate calcium disodium anhydrous, edetic acid. In some of the preferred embodiments, a chelating agent is edetate disodium (disodium EDTA).

The term “pharmaceutically acceptable excipients” or “an excipient” as used herein refers to such pharmaceutically acceptable excipients which are known to those skilled in the art for the purpose of preparing parenteral or injectable pharmaceutical compositions. Such pharmaceutically acceptable excipients without limitation include stabilizing agents, chelating agents, anti-oxidants, surfactants and the like or combinations thereof. Such pharmaceutically acceptable excipients can be used in an amount which provides the compositions of the present invention desired property for which they are intended to use.

The specifically mentioned pharmaceutically acceptable excipients in the foregoing paragraphs are intended to be exemplary and not exhaustive of specific excipients that may be used in the practice of the disclosed invention. It is further understood that more than one of any particular type of excipient may be used in the compositions described herein. For example, the compositions may include more than one solvent/co-solvent, more than one antioxidant, more than one buffering agent and/or more than one pH adjusting/modifying agent, more than one preservative etc. Also, a single excipient may provide multiple functions, as mentioned hereinabove.

As used herein, “comprises” “comprising”, “containing” and “having” and the like can have the meaning ascribed to them in patent law and can mean “includes”, “including” and the like, and are generally interpreted to be open ended terms. The terms “consisting of” or “consists of” are closed terms, and include only the components, structures, steps, or the like specifically listed in conjunction with such terms, as well as that which is in accordance with patent law. “Consisting essentially of” or “consists essentially of” have the meaning generally ascribed to them by patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. When using an open ended term, like “comprising” or “including” it is understood that direct support should be afforded also to “consisting essentially of” language as well as “consisting of” language as if stated explicitly and vice versa. In essence, use of one of these terms in the specification provides support for all of the others. The term “comprise/comprises/comprising” as used herein mean that other ingredients, steps, etc. are optionally present. When reference is made herein to a method comprising two or more defined steps, the steps can be carried in any order or simultaneously (except where the context excludes that possibility), and the method can include one or more steps which are carried out before any of the defined steps, between two of the defined steps, or after all of the defined steps (except where the context excludes that possibility).

As used herein, the term “subject” refers to a mammal. Examples of subjects include humans, and may also include other animals such as horses, pigs, cattle, dogs, cats, rats, rabbits, and aquatic mammals.

As used herein, “treat”, “treating” and “treatment”, means the treatment of a disease in a subject, for example, a human, and includes inhibiting the disease (e.g., decreasing its rate of progression); regressing the disease; relieving or decreasing the severity of one or more symptoms of the disease; and/or curing the disease.

As used herein, “prevent”, “preventing”, and “prevention” means the prevention of a disease in a subject, and includes inhibiting initiation of the disease; decreasing a predisposition toward the disease; and/or delaying the onset of at least one symptom of the disease.

In certain embodiments, the liquid compositions of the present invention are stable under storage conditions. As used herein, the terms “stable” or “stability” encompass any characteristic of the composition which may be affected by storage conditions including, without limitation, potency, total impurities, levothyroxine degradation products, specific optical rotation, optical purity, water content, appearance, viscosity, sterility, and color and clarity. The storage conditions which may affect stability include, for example, duration of storage, temperature, humidity, and/or light exposure.

In one of the further embodiments, the present invention provides liquid concentrate compositions of Levothyroxine or its pharmaceutically acceptable salt which are ready to dilute compositions. These compositions may be diluted before administering to the patient with an appropriate diluent such as, for example, WFI (water for injection), 0.9% sodium chloride, or 5% dextrose.

In certain embodiments, stable liquid compositions refer to compositions which retain at least about 90%, or at least about 95%, or at least about 96%, or at least about 98%, of the labelled concentration of Levothyroxine or its pharmaceutically acceptable salt after storage under typical and/or accelerated conditions. In further embodiments, stable liquid compositions refer to less than about 15% (area percent), or less than about 10% (area percent), or less than about 7% (area percent), or less than about 5% (area percent), or less than about 2% (area percent) of Levothyroxine-related impurities are present after storage under typical and/or accelerated conditions. Typical storage conditions include but not limited to 2° C.-8° C., 40° C.±2° C./75±5% RH, 30° C.±2° C./65±5/RH, 25° C.±2° C./40±5% RH, 25° C.±2° C./60±5% RH (RH=relative humidity).

In some embodiments, the liquid compositions of the invention are stable for at least 3 months or more, at least 6 months or more, or at least 12 months or more at refrigerated temperature (e.g., at 5±3° C.). In other embodiments, the liquid compositions of the invention are stable for at least 3 months or more, at least 6 months or more, or at least 12 months or more at room temperature (e.g., at 25±2° C.). In some embodiments, the liquid compositions of the invention are stable for at least 1 month or more, at least 2 months or more, at least 3 months or more, at least 6 months or more, or at least 12 months or more under accelerated conditions (e.g., at 40±2° C.).

The term “degradant”, “impurity”, “degradation impurity” and “related substance” as used herein represents the same meaning and can be used interchangeably.

Methods for determining the stability of the liquid compositions of the invention with respect to a given parameter are well-known to those of skill in the art. For example, individual impurities and total impurities can be assessed by high-performance liquid chromatography (HPLC) or thin layer chromatography (TLC). Unless otherwise indicated to the contrary, a percentage amount of liothyronine, other individual impurities, or total impurities reported herein in the liquid compositions are determined by a peak area percent method using HPLC.

When the liquid compositions comprise Levothyroxine sodium, the Levothyroxine sodium can be present in the compositions in any suitable concentration. Typically, Levothyroxine sodium can be present in the compositions at a concentration of about 500 mcg/mL or less, for example, about 450 mcg/mL or less, about 400 mcg/mL or less, about 350 mcg/mL or less, about 300 mcg/mL or less, about 250 mcg/mL or less, about 200 mcg/mL or less, or about 150 mcg/mL or less.

Alternatively, Levothyroxine sodium can be present in the compositions at a concentration of about 5 mcg/mL (micrograms/milliliter) or more, for example, about 10 mcg/mL or more, about 15 mcg/mL or more, about 20 mcg/mL or more, about 25 mcg/mL or more, about 30 mcg/mL or more, about 35 mcg/mL or more, about 40 mcg/mL or more, or about 45 mcg/mL or more.

Levothyroxine sodium can be present in the formulation in a concentration bounded by any two of the aforementioned endpoints. For example, levothyroxine sodium can be present in the formulation in a concentration of about 5 mcg/mL to about 500 mcg/mL, for example, about 10 mcg/mL to about 450 mcg/mL, about 15 meg/mL to about 400 mcg/mL, about 20 mcg/mL to about 350 mcg/mL, about 25 mcg/mL to about 300 mcg/mL, about 30 mcg/mL to about 300 mcg/mL, about 35 mcg/mL to about 300 mcg/mL, about 40 mcg/mL to about 300 mcg/mL, about 45 mcg/mL to about 300 mcg/mL, or about 50 mcg/mL to about 250 mcg/mL, or about 20 mcg/mL to about 100 mcg/mL.

In certain embodiments, levothyroxine sodium can be present at a concentration of about 500 mcg/mL or more, about 1000 mcg/mL or more, about 1500 mcg/mL or more, about 2000 mcg/mL or more or about 2500 mcg/mL or more.

In an embodiment, levothyroxine sodium is present at a concentration of about 100 mcg/mL. In another embodiment, levothyroxine sodium is present at a concentration of about 200 mcg/mL. In yet another embodiment, levothyroxine sodium is present at a concentration of about 500 mcg/mL. In yet another embodiment, levothyroxine sodium is present at concentration of about 500 mcg/mL or more.

In an embodiment, levothyroxine sodium is present at a concentration of about 20 mcg/mL. In another embodiment, levothyroxine sodium is present at a concentration of about 40 mcg/mL. In yet another embodiment, levothyroxine sodium is present at a concentration of about 100 mcg/mL.

The liquid compositions can be provided in any suitable volume. In some embodiments, the volume of the formulation is about 0.1 mL or more, about 0.2 mL or more, about 0.5 mL or more, about 1.0 mL or more, about 3 mL or more or about 5.0 mL or more. In some embodiments, the volume of the liquid compositions are about 0.2 to about 1.0 mL. In some embodiments, the volume of the liquid compositions is about 5 mL. One of ordinary skill in the art can readily select an appropriate container based upon the volume of the formulation.

As used herein, the term “about” is synonymous with “approximately” and is used to provide flexibility to a numerical value or range endpoint by providing that a given value may be “a little above” or “a little below” the value stated. “About” can mean, for example, within 3 or more than 3 standard deviations. “About” can mean within a percentage range of a given value. For example, the range can be ±1%, ±5%, ±10%, ±20%, ±30%, ±40% or ±50% of a given value. “About” can mean with an order of magnitude of a given value, for example, within 2-fold, 3-fold, 4-fold or 5-fold of a value. However, it is to be understood that even when a numerical value is accompanied by the term “about” in this specification, that express support shall be provided at least for the exact numerical value as well as though the term “about” were not present.

The use of the terms “a” and “an” and “the” and similar referents in the context of describing the invention (especially in the context of the claims) are to be construed to cover both the singular and the plural, unless otherwise indicated herein or clearly contradicted by context.

As used herein, terms “parenteral compositions”, “liquid compositions”, “parenteral compositions comprising Levothyroxine” refer to formulations that contain Levothyroxine or its pharmaceutically acceptable salt in dissolved or solubilised form. As used herein, terms “liquid concentrate compositions of Levothyroxine”, “ready to dilute liquid concentrate” refer to formulations that contain Levothyroxine or its pharmaceutically acceptable salt in dissolved or solubilised form and are intended to be used upon dilution in suitable diluents. As used herein, terms “ready to use liquid compositions” refer to formulations that contain Levothyroxine or its pharmaceutically acceptable salt in dissolved or solubilized form and are intended to be used as such without dilution.

The certain embodiments, the present invention also provides a container comprising a liquid composition comprising Levothyroxine or its pharmaceutically acceptable salt, one or more solvents/co-solvents and optionally one or more buffering agents or any other optional components. In certain embodiments, the container is a vial, an ampoule, a bag, a bottle, a cartridge, or a syringe. In some embodiments, the container, the composition, or both the container and the composition are sterile. Preferably, the container is sealed by way of a closure, such as a stopper, plunger, and/or tip-cap.

The container and closure can be made of glass, plastic, and/or rubber. One or more surfaces of the container and/or closure can be treated with a compound to limit reactivity with one or more components of the formulation. In some embodiments, the container and/or closure are treated with silicon. In other embodiments, the container is treated with ammonium sulfate ((NH₄)₂SO₄). The container can be clear or opaque, and can be any color. In some embodiments, the container is flint colored. In other embodiments, the container is amber colored.

In certain embodiments, the invention provides a pre-filled syringe containing a liquid composition of the invention described herein. In certain embodiments, a syringe according to the invention is a component of an auto-injector.

The general formula of the liquid parenteral compostions of the present invention can be provided as follows:

| Ingredient | F-1 | F-2 | F-3 | F-4 | F-5 | F-6 |
|---|---|---|---|---|---|---|
| Levothyroxine or | 20 mcg to | 20 mcg to | 20 mcg to | 20 mcg to | 20 mcg to | 20 mcg to |
| its pharmaceutically | 2500 mcg | 2500 mcg | 2500 mcg | 2500 mcg | 2500 mcg | 2500 mcg |
| acceptable salt | | | | | | |
| Solvent(s)/co-solvent(s) | Q.S. | Q.S. | Q.S. | Q.S. | Q.S. | Q.S. |
| Antioxidant | — | — | — | — | — | Q.S. to prevent, |
| | | | | | | oxidation/ |
| | | | | | | degradation of |
| | | | | | | the drug |
| Buffering agent(s) | — | Q.S. to adjust | Q.S. to adjust | Q.S. to adjust | Q.S. to adjust | Optionally used |
| | | the pH between | the pH between | the pH between | the pH between | to adjust the pH |
| | | about 10.0 and | about 3.0 and | about 3.0 and | about 3.0 and | between about |
| | | about 11.0 | about 5.0 | about 5.0 | about 5.0 | 3.0 and about 5.0 |
| pH adjusting/modifying | — | — | Q.S. to adjust | Q.S. to adjust | Q.S. to adjust | Q.S. to adjust |
| agent(s) | | | the pH between | the pH between | the pH between | the pH between |
| | | | about 10.0 and | about 10.0 and | about 10.0 and | about 10.0 and |
| | | | about 11.0 | about 11.0 | about 11.0 | about 11.0 |
| Preservative(s) | — | — | — | — | Q.S. to preserve | — |
| | | | | | the composition | |
| Vehicle(s)/carrier(s) | — | — | — | Q.S. | Q.S. | Q.S. |

Q.S. = quantity sufficient

The liquid compositions according to the invention are suitable for administration to a subject to treat or prevent a disease or condition. Preferably, the subject is a mammal. More preferably, the mammal is a human. Preferably, the disease or condition is a disease or condition that is treatable by the administration of Levothyroxine or a pharmaceutically acceptable salt thereof such as hypothyroidism. In some embodiments, the condition is myxedema coma.

The liquid compositions prepared according to the present invention exhibit unexpectedly good stability profile that makes these compositions suitable for use in the industry.

### EXAMPLES

The liquid compositions of the present invention are explained in more detail with reference to the following examples. These examples are provided by way of illustration only and should not be construed as to limit the scope or spirit of the claims in any manner.

### Example-1: Preparation of Ready to Dilute Liquid Concentrate Formulations of Levothyroxine

| Sr. | | Quantity | | Quantity | | Quantity | |
|---|---|---|---|---|---|---|---|
| No. | Ingredients | per vial | | per vial | | per vial | |
| 1 | Levothyroxine | 500 | mcg | 200 | mcg | 100 | mcg |
| | sodium | | | | | | |
| 2 | Triethanolamine | 7.91 | mg | 7.91 | mg | 7.91 | mg |

| | | | | |
|---|---|---|---|---|
| 3 | Glycerin | Q.S. to | Q.S. to | Q.S. to |
| | | 0.165 mL | 0.165 mL | 0.165 mL |

Q.S. = Quantity sufficient

### Example-2: Preparation of Ready to Dilute Liquid Concentrate Formulations of Levothyroxine

| Sr. | | Quantity | | Quantity | | Quantity | |
|---|---|---|---|---|---|---|---|
| No. | Ingredients | per vial | | per vial | | per vial | |
| 1 | Levothyroxine | 500 | mcg | 200 | mcg | 100 | mcg |
| | sodium | | | | | | |
| 2 | Triethanolamine | 2.10 | mg | 2.10 | mg | 2.10 | mg |

| | | | | |
|---|---|---|---|---|
| 3 | Glycerin | Q.S. to | Q.S. to | Q.S. to |
| | | 0.2 mL | 0.2 mL | 0.2 mL |

Q.S. = Quantity sufficient

### Example-3: Preparation of Ready to Dilute Liquid Concentrate Formulations of Levothyroxine

| Sr. | | Quantity | Quantity | Quantity |
|---|---|---|---|---|
| No. | Ingredients | per vial | per vial | per vial |
| 1 | Levothyroxine | 500 mcg | 200 mcg | 100 mcg |
| | sodium | | | |
| 2 | Triethanolamine | 0.1-20 mg | 0.1-20 mg | 0.1-20 mg |
| 3 | Glycerin | Q.S. to | Q.S. to | Q.S. to |
| | | 0.2 mL | 0.2 mL | 0.2 mL |

Q.S. = Quantity sufficient

Process of Preparing Examples-1 to 3:
1. Take about 80% glycerin in suitable vessel;
2. Add suitable quantity of triethanolamine in step (1) to adjust the desired pH and mix until it gets dissolved;
3. Add required quantity of Levothyroxine sodium in step (2) and mix until it gets dissolved; and
4. Adjust the volume to required batch size and mix till homogenous solution is formed.

### Example-4: Preparation of Ready to Dilute Liquid Concentrate Formulations of Levothyroxine

| Sr. | | Quantity | Quantity | Quantity |
|---|---|---|---|---|
| No. | Ingredients | per vial | per vial | per vial |
| 1 | Levothyroxine | 500 mcg | 200 mcg | 100 mcg |
| | sodium | | | |
| 2 | Ethanol | 0.001-0.08 mL | 0.001-0.08 mL | 0.001-0.08 mL |
| 3 | Glycerin | Q.S. to | Q.S. to | Q.S. to |
| | | 0.2 mL | 0.2 mL | 0.2 mL |

Process of Preparing Example-4:
1. Take about 80% glycerin in suitable vessel;
2. Add required quantity of ethanol in step (1) and mix until it gets dispersed
3. Add required quantity of Levothyroxine sodium in step (2) and mix until it gets dissolved;
4. Adjust the volume to required batch size and mix till homogenous solution is formed.

### Examples-5 to 8: Preparation of Ready to Dilute Liquid Concentrate Formulations of Levothyroxine

| | Role of | Concentration per mL | | | | | | | |
|---|---|---|---|---|---|---|---|---|---|
| Ingredients | ingredients | Example-5 | | Example-6 | | Example-7 | | Example-8 | |
| Levothyroxine | Active | 500 | mcg | 500 | mcg | 500 | mcg | 2500 | mcg |
| sodium | ingredient | | | | | | | | |
| Glycerin | Solvent/co- | 756 | mg | 500 | mg | 500 | mg | 756 | mg |
| | solvent | | | | | | | | |
| Citric acid | Antioxidant | 0.5 | mg | 0.5 | mg | 1 | mg | 1 | mg |

| | | | | | |
|---|---|---|---|---|---|
| Sodium | pH adjusting | Q.S. to adjust/ | Q.S. to adjust/ | Q.S. to adjust/ | Q.S. to adjust/ |
| hydroxide | agent | maintain the pH | maintain the pH | maintain the pH | maintain the pH |
| | | between about 8.0 | between about 8.0 | between about 8.0 | between about 8.0 |
| | | and about 12.0 | and about 12.0 | and about 12.0 | and about 12.0 |
| Water for | Vehicle/Carrier | Q.S. to 1 mL | Q.S. to 1 mL | Q.S. to 1 mL | Q.S. to 1 mL |
| injection | | | | | |

| | | | | | | | | |
|---|---|---|---|---|---|---|---|---|
| Fill volume per vial | 1 | mL | 1 | mL | 1 | mL | 0.2 | mL |

Process of Preparing Examples-5 to 8:
1. Mix required quantities of glycerin and water for injection in a suitable vessel;
2. Add required quantity of citric acid in step (1);
3. Adjust pH of step (2) between about 8.0 and about 12.0 with sodium hydroxide;
4. Add and dissolve required quantity of Levothyroxine sodium in step (3); and
5. Adjust the volume with water for injection to required batch size.

The ready to dilute liquid concentrate formulations of Levothyroxine prepared according to the present invention as exemplified in Examples-5 to 8 were tested for its physical stability. The liquid concentrate formulations were kept at various storage conditions such as 40° C.±2° C. and 75%±5% RH (relative humidity), 25° C.±2° C. and 60%±5% RH (relative humidity). The amount of impurities (related substances or degradation products) present in the compositions were tested by HPLC at initial level and after three months after stored the formulations of the present invention at above mentioned conditions. The results are summarized as under.

### Stability Study Results of Example-5

| | | 40° C./75% | 25° C./60% |
|---|---|---|---|
| Test parameters | Initial | RH 3 months | RH 3 months |
| Description | A clear | A clear | A clear |
| | colorless | colorless | colorless |
| | solution | solution | solution |
| Assay | 95.00% | 86.00% | 92.90% |
| pH | 10.0 | 8.8 | 8.6 |

Related substances

| | | | |
|---|---|---|---|
| Liothyronine | 0.02% | 0.99% | 0.18% |
| T4-hydroxyacetic | ND | ND | ND |
| acid/β-hydroxy T4 | | | |
| T4-aIdehyde | ND | ND | ND |
| T4-benzoic acid | 0.12% | 0.81% | 0.25% |
| RRT-0.11 | ND | 1.28% | 0.34% |
| RRT-0.85 | ND | 0.30% | 0.36% |
| RRT-0.94 | ND | 0.62% | 0.49% |
| RRT-0.99 | 0.12% | 0.16% | 0.16% |
| RRT-1.79 | ND | ND | 0.11% |
| RRT-1.82 | 0.05% | 0.56% | 0.14% |
| RRT-1.83 | ND | 0.20% | 0.09% |
| RRT-1.89 | 0.21% | 1.30% | 1.00% |
| Total impurities | 0.80% | 7.00% | 3.62% |

ND = not detected

### Stability Study Results of Example-6

| | | 40° C./75% | 25° C./60% |
|---|---|---|---|
| Test parameters | Initial | RH 3 months | RH 3 months |
| Description | A clear | A clear | A clear |
| | colorless | colorless | colorless |
| | solution | solution | solution |
| Assay | 97.30% | 90.40% | 97.00% |
| pH | 10.3 | 9.2 | 9.2 |

Related substances

| | | | |
|---|---|---|---|
| Liothyronine | 0.02% | 1.04% | 0.15% |
| T4-hydroxyacetic | ND | ND | ND |
| acid/β-hydroxy T4 | | | |
| T4-aldehyde | ND | ND | ND |
| T4-benzoic acid | 0.13% | 0.65% | 0.25% |
| RRT-0.11 | ND | 2.23% | 0.50% |
| RRT-0.85 | ND | 0.24% | 0.29% |
| RRT-0.94 | ND | 0.16% | 0.08% |
| RRT-1.82 | ND | 0.30% | 0.09% |
| RRT-1.83 | ND | 0.14% | 0.10% |
| RRT-1.87 | ND | ND | 0.07% |
| RRT-1.91 | 0.13% | ND | ND |
| RRT-1.92 | ND | 0.19% | 0.56% |
| RRT-1.95 | ND | 0.50% | ND |
| RRT-1.96 | ND | 0.19% | ND |
| RRT-1.99 | ND | 0.07% | ND |
| RRT-2.07 | ND | 0.17% | 0.09% |
| Total impurities | 0.57% | 6.10% | 2.42% |

ND = not detected

### Stability Study Results of Example-7

| | | 40° C./75% | 25° C./60% |
|---|---|---|---|
| Test parameters | Initial | RH 3 months | RH 3 months |
| Description | A clear | A clear | A clear |
| | colorless | colorless | colorless |
| | solution | solution | solution |
| Assay | 98.00% | 91.50% | 95.10% |
| pH | 8.4 | 8.93 | 8.94 |

Related substances

| | | | |
|---|---|---|---|
| Liothyronine | 0.03% | 0.86% | 0.19% |
| T4-hydroxyacetic | ND | ND | ND |
| acid/β-hydroxy T4 | | | |
| T4-aldehyde | ND | ND | ND |
| T4-benzoic acid | 0.19% | 0.74% | 0.35% |
| RRT-0.11 | 0.05% | 0.87% | 0.24% |
| RRT-0.83 | ND | 0.11% | ND |
| RRT-0.95 | ND | 0.14% | 0.10% |
| RRT-1.70 | 0.08% | ND | ND |
| RRT-1.79 | 0.08% | ND | ND |
| RRT-1.84 | 0.08% | ND | ND |
| RRT-1.88 | 0.09% | ND | ND |
| RRT-1.94 | ND | 0.43% | 0.13% |
| RRT-1.97 | ND | 0.13% | 0.07% |
| RRT-2.00 | ND | 0.08% | ND |
| RRT-2.07 | ND | 0.25% | 0.09% |
| RRT-2.18 | ND | 0.07% | ND |
| RRT-2.23 | ND | 0.05% | 0.25% |
| RRT-2.36 | ND | ND | 0.07% |
| Total impurities | 0.64% | 4.06% | 1.78% |

ND = not detected

### Stability Study Results of Example-8

| | | 40° C./75% | 25° C./60% |
|---|---|---|---|
| Test parameters | Initial | RH 3 months | RH 3 months |
| Description | A clear | A clear | A clear |
| | colorless | colorless | colorless |
| | solution | solution | solution |
| Assay | 98.40% | 90.40% | 97.00% |
| pH | 11.3 | 11.7 | 11.6 |

Related substances

| | | | |
|---|---|---|---|
| Liothyronine | 0.03% | 0.25% | 0.05% |
| T4-hydroxyacetic | ND | ND | ND |
| acid/β-hydroxy T4 | | | |
| T4-aldehyde | ND | ND | ND |
| T4-benzoic acid | 0.21% | 0.45% | 0.27% |
| RRT-0.11 | ND | 0.27% | 0.05% |
| RRT-0.64 | ND | 0.06% | ND |
| RRT-0.83 | ND | 1.90% | 0.27% |
| RRT-0.97 | ND | 0.07% | ND |
| RRT-1.53 | ND | 0.12% | ND |
| RRT-1.77 | 0.07% | ND | ND |
| RRT-1.83 | 0.07% | ND | ND |
| RRT-1.88 | 0.18% | ND | ND |
| RRT-1.92 | ND | 0.09% | ND |
| RRT-1.94 | ND | 0.48% | 0.16% |
| RRT-1.96 | ND | 0.06% | ND |
| RRT-2.00 | ND | 0.09% | 0.07% |
| RRT-2.04 | ND | 0.27% | ND |
| RRT-2.05 | ND | ND | 0.05% |
| RRT-2.06 | ND | ND | 0.19% |
| RRT-2.13 | 0.04% | 0.41% | 0.20% |
| RRT-2.19 | ND | 0.10% | ND |
| RRT-2.23 | ND | 0.09% | ND |
| RRT-2.25 | ND | 0.06% | ND |
| Total impurities | 0.72% | 5.29% | 1.66% |

ND = not detected

### Example-9: Preparation of Ready to Dilute Liquid Concentrate Formulations of Levothyroxine

| Sr No | Ingredients | Quantity per mL |
|---|---|---|
| 1 | Levothyroxine | 500 mcg to 2500 mcg |
| | sodium | |
| 2 | Citric acid | Q.S. to prevent |
| | monohydrate | oxidation/degradation |
| 3 | Sodium hydroxide | Q.S. to adjust/maintain pH |
| | | between about 8.0 to |
| | | about 12.0 |
| 4 | Glycerin | Q.S. to 1.0 mL |

Q.S. = quantity sufficient

Process of Preparing Example-9:
1. Take required quantity of glycerin in suitable vessel;
2. Add required quantity of citric acid in step (1);
3. Add sodium hydroxide solution in step (2) in quantity sufficient to adjust the pH between about 8.0 and about 12.0;
4. Add and dissolve Levothyroxine sodium in step (3); and
5. Adjust the volume with glycerin to desired batch size.

### Example-10: Preparation of Ready to Dilute Liquid Concentrate Formulations of Levothyroxine

| Sr. No. | Ingredient | Quantity per mL |
|---|---|---|
| 1 | Levothyroxine | 500 mcg to 2500 mcg |
| | sodium | |
| 2 | Glycerin | 756.0 mg |
| 3 | Citric acid | Q.S. to prevent |
| | monohydrate | oxidation/degradation |
| 4 | Sodium hydroxide | Q.S. to adjust/maintain pH |
| | | between about 8.0 and |
| | | about 12.0 |
| 5 | Water for | Q.S. to 1.0 mL |
| | injection | |

Q.S. = quantity sufficient

Process of Preparing Example-10:
1. Take required quantity of glycerin in suitable vessel;
2. Add required quantity of water for injection in step (1) and mix until uniform mixture is formed;
3. Add required quantity of citric acid in step (2);
4. Add sodium hydroxide solution in step (3) in a quantity sufficient to adjust the pH between about 8.0 and about 12.0;
5. Add and dissolve required quantity of Levothyroxine sodium in step (4); and
6. Adjust the volume with water for injection to the desired batch size.

### Example-11: Preparation of Ready to Dilute Liquid Concentrate Formulations of Levothyroxine

| Sr. No. | Ingredient | Quantity per mL | |
|---|---|---|---|
| 1 | Levothyroxine sodium | 500 mcg to 2500 mcg | |
| 2 | Glycerin | 756.0 | mg |
| 3 | Sodium methyl paraben | 7.5 | mg |

| | | |
|---|---|---|
| 4 | Citric acid monohydrate | Q.S. to prevent |
| | | oxidation/degradation |
| 5 | Sodium hydroxide | Q.S. to adjust/maintain the pH |
| | | between about 8.0 and 12.0 |
| 6 | Water for injection | Q.S. to 1.0 mL |

Q.S. = quantity sufficient

Process of Preparing Example 11:
1. Take required quantity of glycerin in suitable vessel;
2. Add required quantity of water for injection in step (1) and mix until uniform mixture is formed;
3. Add and dissolve required quantity of sodium methyl paraben in step (2);
4. Add required quantity of citric acid in step (3);
5. Add and dissolve required quantity of Levothyroxine sodium in step (4);
6. Add sodium hydroxide solution in step (5) in a quantity sufficient to adjust the pH between about 8.0 and about 12.0; and
7. Adjust the volume with water for injection to the desired batch size.

### Examples 12-20: Preparation of Ready to Dilute Liquid Concentrate Formulations of Levothyroxine

| | | Quantity per mL | | | | | | | | | |
|---|---|---|---|---|---|---|---|---|---|---|---|
| Sr. No. | Ingredient | Ex-12 | | Ex-13 | | Ex-14 | | Ex-15 | | Ex-16 | |
| 1 | Levothyroxine sodium | 500 | mcg | 500 | mcg | 500 | mcg | 500 | mcg | 500 | mcg |
| 2 | Glycerin | 500 | mg | 500 | mg | 500 | mg | 500 | mg | 500 | mg |
| 3 | Citric acid | 1 | mg | 1 | mg | 1 | mg | 1 | mg | 1 | mg |

| 4 | Sodium hydroxide | Q.S.^{#} | | Q.S.^{#} | | Q.S.^{#} | | Q.S.^{#} | Q.S.^{#} |
|---|---|---|---|---|---|---|---|---|---|
| 5 | Sodium iodide | 0.14-0.7 | mg | 0.14-0.7 | mg | 0.14-0.7 | mg | — | — |

| | | | | | | | | |
|---|---|---|---|---|---|---|---|---|
| 6 | Disodium EDTA | — | — | — | 0.05-2 | mg | 0.05-2 | mg |

| | | | | | | |
|---|---|---|---|---|---|---|
| 7 | Water for injection | Q.S. to 1 mL | Q.S. to 1 mL | Q.S. to 1 mL | Q.S. to 1 mL | Q.S. to 1 mL |

| | | | | | | | | | | |
|---|---|---|---|---|---|---|---|---|---|---|
| Fill volume per vial | 0.2 | mL | 0.4 | mL | 1.0 | mL | 0.2 | mL | 0.4 | mL |

| | | Quantity per mL | | | | | | | |
|---|---|---|---|---|---|---|---|---|---|
| Sr. No. | Ingredient | Ex-17 | | Ex-18 | | Ex-19 | | Ex-20 | |
| 1 | Levothyroxine sodium | 500 | mcg | 500 | mcg | 500 | mcg | 500 | mcg |
| 2 | Glycerin | 500 | mg | 500 | mg | 500 | mg | 500 | mg |
| 3 | Citric acid | 1 | mg | 1 | mg | 1 | mg | 1 | mg |
| 4 | Sodium hydroxide | Q.S.^{#} | | Q.S.^{#} | | Q.S.^{#} | | Q.S.^{#} | |
| 5 | Sodium iodide | — | | 0.14-0.7 | mg | 0.14-0.7 | mg | 0.14-1.7 | mg |
| 6 | Disodium EDTA | 0.05-2 | mg | 0.05-2 | mg | 0.05-2 | mg | 0.05-2 | mg |

| | | | | | |
|---|---|---|---|---|---|
| 7 | Water for injection | Q.S. to 1 mL | Q.S. to 1 mL | Q.S. to 1 mL | Q.S. to 1 mL |

| | | | | | | | | |
|---|---|---|---|---|---|---|---|---|
| Fill volume per vial | 1.0 | mL | 0.2 | mL | 0.4 | mL | 1.0 | mL |

Q.S.^{#} = quantity sufficient to adjust/maintain pH between about 8.0 and about 12.0
Q.S. = quantity sufficient

### Examples 21-23: Preparation of Ready to Use Liquid Formulations of Levothyroxine

| | | Quantity per mL | | | | | |
|---|---|---|---|---|---|---|---|
| Sr. No. | Ingredient | Example-21 | | Example-22 | | Example-23 | |
| 1 | Levothyroxine | 20/40/100 | | 20/40/100 | | 20/40/100 | |
| | sodium | mcg | | mcg | | mcg | |
| 2 | Glycerin | 100 | mg | 100 | mg | 100 | mg |
| 3 | Citric acid | 0.2 | mg | 0.2 | mg | 0.2 | mg |

| | | | | |
|---|---|---|---|---|
| 4 | Sodium hydroxide | Q.S.^{#} | Q.S.^{#} | Q.S.^{#} |
| 5 | Sodium iodide | 0.14-0.7 mg | — | 0.14-0.7 mg |
| 6 | Disodium EDTA | — | 0.05-0.2 mg | 0.05-0.2 mg |
| 7 | Water for | Q.S. to 1 mL | Q.S. to 1 mL | Q.S. to 1 mL |
| | Injection | | | |

| | | | | | | |
|---|---|---|---|---|---|---|
| Fill volume per vial | 5 | mL | 5 | mL | 5 | mL |

Q.S.^{#} = quantity sufficient to adjust/maintain pH between about 8.0 and about 12.0

Process of Preparing Examples 12-23:
1. Mix required quantities of glycerin and water for injection in a suitable vessel;
2. Add required quantity of citric acid in step (1);
3. Adjust pH of step (1) with sodium hydroxide between about 8.0 and about 12.0;
4. Add and dissolve required quantity of sodium iodide in step (3);
5. Add and dissolve required quantity of levothyroxine sodium in step (4); and
6. Adjust the volume of step (5) with water for injection to desired batch size.

In the above mentioned process, when disodium EDTA is used in the formulations (e.g. in Examples 15-20 & Examples 22 & 23), it is added after the step of dissolving levothyroxine sodium.

The ready to dilute liquid concentrate formulations and ready to use liquid compositions of Levothyroxine prepared according to the present invention as exemplified in Examples-12, 14, 21 and 23 were tested for its physical stability. The liquid formulations were stored at 60° C. for 10 days. The amount of impurities (related substances or degradation products) present in the compositions were tested by HPLC at initial level and after 10 days. The results are summarized as under.

Stability study results of Examples-12, 14, 21& 23

| | | | | | Example-21 | | | | Example-23 | | | |
|---|---|---|---|---|---|---|---|---|---|---|---|---|
| | | | | | Fill Volume-5 | | Fill Volume-5 | | Fill Volume-5 | | Fill Volume-5 | |
| | Example-12 | | Example-14 | | mL in 5 mL vial | | mL in 10 mL vial | | mL in 5 mL vial | | mL in 10 mL vial | |
| | | 60° C. | | 60° C. | | 60° C. | | 60° C. | | 60° C. | | 60° C. |
| Test Parameters | Initial | (10 d) | Initial | (10 d) | Initial | (10 d) | Initial | (10 d) | Initial | (10 d) | Initial | (10 d) |
| Description | # | # | # | # | # | # | # | # | # | # | # | # |
| pH | 8.96 | 8.88 | 9.10 | 9.06 | 7.86 | 8.36 | 7.96 | 8.51 | 7.82 | 8.14 | 7.95 | 8.21 |

Related Substances (in %)

| | | | | | | | | | | | | |
|---|---|---|---|---|---|---|---|---|---|---|---|---|
| Liothyronine | 0.05 | 0.33 | 0.05 | 0.33 | 0.03 | 0.58 | 0.02 | 0.49 | 0.05 | 0.82 | 0.05 | 0.80 |
| T4-hydroxyacetic | ND | ND | ND | ND | ND | ND | ND | ND | ND | ND | ND | ND |
| acid/β-hydroxy-T4 | | | | | | | | | | | | |
| T4-aldehyde | ND | ND | ND | ND | ND | ND | ND | ND | ND | ND | ND | ND |
| T4-benzoic acid | 0.29 | 0.42 | 0.28 | 0.41 | 0.17 | 0.22 | 0.17 | 0.51 | 0.21 | 0.27 | 0.21 | 0.38 |
| RRT-0.09 | 0.07 | ND | 0.08 | ND | 0.07 | ND | 0.07 | ND | 0.07 | ND | 0.06 | ND |
| RRT-0.09 | 0.07 | 0.04 | 0.07 | 0.04 | 0.09 | 0.05 | 0.08 | ND | 0.08 | 0.07 | 0.08 | ND |
| RRT-0.10 | 0.03 | ND | 0.03 | ND | 0.06 | 0.03 | 0.05 | 0.03 | 0.05 | 0.05 | 0.05 | 0.04 |
| RRT-0.11 | 0.04 | 0.15 | 0.03 | 0.13 | 0.39 | 0.26 | 0.07 | 0.49 | 0.05 | 0.19 | 0.08 | 0.50 |
| RRT-0.13 | ND | ND | 0.03 | 0.03 | 0.16 | 0.02 | ND | ND | ND | ND | ND | ND |
| RRT-0.23 | 0.02 | ND | ND | ND | 0.03 | ND | 0.03 | ND | ND | ND | ND | ND |
| RRT-0.82 | 0.04 | 0.07 | 0.04 | 0.06 | 0.03 | 0.04 | ND | 0.04 | ND | ND | ND | ND |
| RRT-0.94 | 0.11 | 0.15 | 0.10 | 0.13 | 0.04 | 0.06 | 0.04 | 0.05 | 0.04 | 0.05 | 0.04 | 0.06 |
| RRT-1.08 | 0.02 | ND | 0.03 | ND | 0.04 | ND | 0.03 | ND | 0.04 | ND | 0.04 | ND |
| RRT-1.09 | 0.02 | ND | 0.03 | ND | 0.05 | ND | 0.03 | ND | 0.04 | ND | 0.03 | ND |
| RRT-1.21 | ND | 0.02 | ND | ND | ND | ND | ND | ND | ND | ND | ND | ND |
| RRT-1.68 | 0.02 | 0.02 | 0.03 | 0.03 | ND | ND | ND | ND | ND | ND | ND | ND |
| RRT-1.71 | ND | ND | ND | ND | ND | ND | ND | 0.06 | ND | 0.02 | ND | 0.05 |
| RRT-1.76 | 0.04 | 0.06 | 0.04 | 0.06 | 0.02 | 0.03 | 0.02 | 0.04 | 0.03 | 0.04 | 0.03 | 0.04 |
| RRT-1.78 | 0.11 | 0.40 | 0.09 | 0.39 | 0.09 | 0.17 | 0.08 | 0.34 | 0.07 | 0.17 | 0.07 | 0.24 |
| RRT-1.79 | 0.08 | 0.09 | 0.08 | 0.09 | 0.02 | 0.04 | 0.02 | 0.12 | 0.03 | 0.08 | 0.03 | 0.12 |
| RRT-1.82 | 0.04 | 0.03 | 0.04 | 0.03 | ND | 0.10 | ND | ND | ND | 0.02 | ND | ND |
| RRT-1.84 | 0.10 | 0.09 | 0.10 | 0.09 | 0.13 | 0.03 | 0.08 | 0.09 | 0.10 | 0.11 | 0.08 | 0.10 |
| RRT-1.90 | 0.25 | 0.28 | 0.25 | 0.28 | 0.10 | 0.08 | 0.08 | 0.09 | 0.12 | 0.11 | 0.12 | 0.11 |
| RRT-1.93 | ND | ND | ND | ND | ND | ND | ND | ND | 0.02 | ND | 0.02 | ND |
| RRT-2.04 | 0.03 | 0.12 | 0.02 | 0.10 | 0.10 | ND | ND | 0.09 | ND | ND | ND | ND |
| RRT-2.15 | 0.03 | 0.03 | 0.03 | 0.03 | ND | ND | ND | ND | ND | ND | ND | ND |
| RRT-2.16 | 0.03 | 0.04 | 0.03 | 0.04 | ND | ND | ND | ND | ND | ND | ND | ND |
| RRT-2.20 | ND | 0.02 | ND | ND | ND | ND | ND | ND | ND | ND | ND | ND |
| Total impurities | 1.49 | 2.36 | 1.48 | 2.27 | 1.62 | 1.71 | 0.87 | 2.47 | 1.00 | 2.00 | 0.99 | 2.44 |

ND = Not Detected
# = Clear colorless solution

### Example-24: Preparation of Ready to Dilute Liquid Formulations of Levothyroxine

| Sr. No. | Ingredient | Quantity per mL | |
|---|---|---|---|
| 1 | Levothyroxine | 500 | mcg |
| | sodium | | |
| 2 | Glycerin | 500 | mg |
| 3 | Glycine | 3.75 | mg |

| | | |
|---|---|---|
| 4 | Sodium hydroxide | Quantity sufficient to |
| | | adjust/maintain pH between |
| | | about 8.0 and about 12.0 |
| 5 | Water for injection | Quantity sufficient |
| | | to 1 mL |

| | | |
|---|---|---|
| Fill volume per vial | 1 | mL |

Process of Preparing Example-24:
1. In a suitable vessel prepare glycine-sodium hydroxide buffer in required quantity;
2. In a separate vessel take the required quantity of glycerin and add required quantity of glycine buffer into it;
3. Add and dissolve required quantity of levothyroxine sodium; and
4. Adjust the volume with water for injection to desired batch size.

The ready to dilute liquid concentrate formulations of Levothyroxine prepared according to the present invention as exemplified in Example-24 were tested for its physical stability. The liquid formulations were stored at 60° C. for one week. The amount of impurities (related substances or degradation products) present in the compositions were tested by HPLC at initial level and after one week. The results are summarized as under.

Stability Study Results of Example 44

Example-24

| Test parameters | Initial | 60° C. (1 week) |
|---|---|---|
| Description | Clear colorless | Clear colorless |
| | solution | solution |
| pH | 9.91 | 9.88 |
| Assay of Levothyroxine | 100.20% | 97.20% |
| sodium | | |

Related substances

| | | |
|---|---|---|
| Liothyronine | 0.05% | 0.17% |
| T4-hydroxyacetic | ND | ND |
| acid/β-hydroxy T4 | | |
| T4-aldehyde | ND | ND |
| T4-benzoic acid | 0.25% | 0.29% |
| RRT-0.11 | 0.03% | 0.12% |
| RRT-0.21 | — | 0.16% |
| RRT-0.71 | — | 0.02% |
| RRT-0.82 | 0.06% | 0.29% |
| RRT-0.94 | — | 0.03% |
| RRT-1.22 | 0.03% | — |
| RRT-1.55 | — | 0.14% |
| RRT-1.79 | 0.09% | — |
| RRT-1.80 | — | 0.70% |
| RRT-1.81 | 0.10% | 0.09% |
| RRT-1.83 | 0.02% | 0.03% |
| RRT-1.84 | — | 0.04% |
| RRT-1.85 | 0.11% | 0.11% |
| RRT-1.87 | — | 0.02% |
| RRT-1.91 | 0.12% | 0.21% |
| RRT-2.16 | 0.02% | 0.02% |
| RRT-2.17 | 0.02% | 0.04% |
| Total impurities | 0.92% | 1.97% |

ND = Not Detected

It is surprisingly found that the ready to dilute liquid compositions of the present invention when diluted with suitable pharmaceutically acceptable diluent, found stable up to 8 hours or more, up to 12 hours or more, up to 24 hours or more, up to 3 days or more, up to 1 week or more, up to 2 weeks or more, up to 3 weeks or more or up to 1 month or more.

It should be understood that various changes and modifications to the herein described embodiments will be apparent to those skilled in the art. Such changes and modifications can be made without departing from the spirit and scope of the subject matter of the present invention and without diminishing its intended advantages. It is therefore intended that such changes and modifications be covered within the scope of the present invention.

What is claimed is:

1. A liquid parenteral composition of levothyroxine comprising:
about 20 meg to about 2500 mg levothyroxine or its pharmaceutically acceptable salt;
an antioxidant;
a pH adjusting agent; and
a vehicle.

2. The liquid parenteral composition of levothyroxine as claimed in claim 1, wherein said composition optionally comprises an excipient selected from the group consisting of a solvent, a buffering agent, a preservative and a combination thereof.

3. The liquid parenteral composition of levothyroxine as claimed in claim 1, wherein said composition does not include a stabilizing agent.

4. The liquid parenteral composition of levothyroxine as claimed in claim 1, wherein said composition has pH between about 8.0 and about 12.0.

5. The liquid parenteral composition of levothyroxine as claimed in claim 1, wherein said composition is a ready to dilute composition or a ready to use composition.

6. The liquid parenteral composition of levothyroxine as claimed in claim 1, wherein an antioxidant is selected from the group consisting of α-tocopherol acetate, ascorbic acid, citric acid, erythorbic acid, butylated hydroxytoluene (BHT), d-α-tocopherol natural, monothioglycerol, thioglycolic acid, sodium bisulfite, sodium sulfite, sodium metabisulfite, potassium metabisulfite, acetone sodium bisulfite, ascorbyl palmitate, cysteine, d-α-tocopherol synthetic, nordihydroguaiaretic acid, sodium formaldehyde sulfoxylate, sodium thiosulfate, acetylcysteine, ascorbyl palmitate, butylated hydroxyanisole (BHA), cysteine hydrochloride, dithiothreitol, propyl gallate, and thiourea or a combination thereof.

7. The liquid parenteral composition of levothyroxine as claimed in claim 1, wherein a pH adjusting agent is selected form the group consisting of acetic acid, adipic acid, ammonium carbonate, ammonium hydroxide, boric acid, citric acid, fumaric acid, hydrochloric acid, malic acid, nitric acid, propionic acid, potassium bicarbonate, potassium chloride, sodium bicarbonate, sodium borate, sodium carbonate, sodium chloride, sodium hydroxide, sodium proprionate, succinic acid, sulfuric acid, tartaric acid, and triethylamine or a combination thereof.

8. The liquid parenteral composition of levothyroxine as claimed in claim 1, wherein a vehicle is water for injection.

9. The liquid parenteral composition of levothyroxine as claimed in claim 2, wherein a solvent is selected from the group consisting of dichloromethane, acetonitrile, ethyl acetate, acetone, propylene carbonate, glycerin, coconut fatty acid diethanolamide, medium and/or long chain fatty acids or glycerides, monoglycerides, diglycerides, triglycerides, structured triglycerides, soyabean oil, peanut oil, corn oil, corn oil mono glycerides, corn oil di glycerides, corn oil triglycerides, polyethylene glycol, caprylocaproylmacroglycerides, caproyl 90, propylene glycol, propylene glycol esters, polyoxyethylenesorbitan fatty acid esters, polyoxyethylene castor oil derivatives, castor oil, cottonseed oil, olive oil, safflower oil, peppermint oil, coconut oil, palm seed oil, beeswax, oleic acid, methanol, ethanol, isopropyl alcohol, butanol, polyethylene alcohol, acetone, methyl isobutyl ketone, methyl ethyl ketone, N-methylpyrrolidone, dimethylacetamide, dimethylsulfoxide, and dimethylisosorbide or a combination thereof.

10. The liquid parenteral composition of levothyroxine as claimed in claim 2, wherein a buffering agent is selected from the group consisting of arginine, alanine, histidine, glycine, lysine, citrate, glutamate, bicarbonate, tartrate, benzoate, lactate, gluconate, triethanolamine, trolamine, acetate, meglumine, borate, phosphate, ammonium phosphate, diethanolamine, potassium acetate, potassium citrate, potassium metaphosphate, potassium phosphate, sodium acetate, sodium citrate, sodium glycolate, sodium lactate and sodium phosphate or a combination thereof.

11. The liquid parenteral composition of levothyroxine as claimed in claim 2, wherein a preservative is selected from the group consisting of ethanol, chlorobutanol, phenoxyethanol, potassium benzoate, benzyl alcohol, benzoic acid, potassium sorbate, sorbic acid, benzalkonium chloride, benzethonium chloride, cetrimonium bromide, cetylpyridinium chloride, bronopol, chlorbutol, chlorocresol, cresol, butylparaben or salt thereof methylparaben or salt thereof, propylparaben or salt thereof, ethylparaben or salt thereof phenol, thymol, phenylethanol, sodium benzoate, propylene glycol, glycerin, and chloroform or a combination thereof.

12. The liquid parenteral composition of levothyroxine as claimed in claim 1 for use in the thyroid hormone replacement therapy in cases of reduced or absent thyroid function.

13. A liquid parenteral composition of levothyroxine comprising:
about 20 mcg to about 2500 mcg levothyroxine or its pharmaceutically acceptable salt;
an antioxidant selected from α-tocopherol acetate, ascorbic acid, citric acid or its salts, erythorbic acid, butylated hydroxytoluene (BHT), d-α-tocopherol natural, monothioglycerol, thioglycolic acid, sodium bisulfite, sodium sulfite, sodium metabisulfite, potassium metabisulfite, acetone sodium bisulfite, ascorbyl palmitate, cysteine or its salts, d-α-tocopherol synthetic, nordihydroguaiaretic acid, sodium formaldehyde sulfoxylate, sodium thiosulfate, acetylcysteine, ascorbyl palmitate, butylated hydroxyanisole (BHA), dithiothreitol, propyl gallate, thiourea and combinations thereof;
a pH adjusting agent selected from sodium hydroxide, potassium hydroxide, calcium hydroxide, sodium carbonate, sodium bicarbonate, potassium carbonate, potassium bicarbonate, calcium carbonate, magnesium carbonate, lithium carbonate, cesium carbonate and combinations thereof;
a solvent selected from propylene glycol, polyethylene alcohol, propylene glycol esters, polyethylene glycols, glycerin and combinations thereof;
optionally a stabilizing agent selected from salts of iodide;
optionally a chelating agent selected from edetate disodium (disodium EDTA), edetate disodium (disodium EDTA) anhydrous, edetate sodium, edetate calcium disodium, edetate calcium disodium anhydrous, edetic acid and combinations thereof; and
a vehicle selected from water for injection.

14. The liquid parenteral composition of levothyroxine as claimed in claim 13, wherein said composition has pH between about 8.0 and about 12.0.

15. A liquid parenteral composition of levothyroxine comprising:
about 20 meg to about 2500 mcg levothyroxine or its pharmaceutically acceptable salt;
a buffering agent;
a solvent;
a vehicle; and
optionally a pH adjusting agent.

16. The liquid parenteral composition of levothyroxine as claimed in claim 15, wherein said composition has pH between about 8.0 and about 12.0.

17. The liquid parenteral composition of levothyroxine as claimed in claim 15, wherein a buffering agent is selected from arginine, alanine, histidine, glycine, lysine, citrate, glutamate, bicarbonate, tartrate, benzoate, lactate, gluconate, triethanolamine, trolamine, acetate, meglumine, borate, phosphate, ammonium phosphate, diethanolamine, potassium acetate, potassium citrate, potassium metaphosphate, potassium phosphate, sodium acetate, sodium citrate, sodium glycolate, sodium lactate, sodium phosphate and combinations thereof.

18. The liquid parenteral composition of levothyroxine as claimed in claim 15, wherein a solvent is selected from propylene glycol, polyethylene alcohol, propylene glycol esters, polyethylene glycols, glycerin and combinations thereof.

19. The liquid parenteral composition of levothyroxine as claimed in claim 15, wherein a vehicle is water for injection.

20. The liquid parenteral composition of levothyroxine as claimed in claim 15, wherein a pH adjusting agent is selected from sodium hydroxide, potassium hydroxide, calcium hydroxide, sodium carbonate, sodium bicarbonate, potassium carbonate, potassium bicarbonate, calcium carbonate, magnesium carbonate, lithium carbonate, cesium carbonate and combinations thereof.
